(19) United States
(12) Plant Patent Application Publication
(10) Pub. No.: US 2009/0049575 P1
(43) Pub. Date: Feb. 19, 2009

(54) Interspecific hybidization of Gardenia jasminoides Ellis and G. thunbergia L.

(76) Inventor: Allyn Cook, Gainesville, FL (US)

Correspondence Address: ALLYN COOK, 109 SW 101 COURT, GAINESVILLE, FL, 32607, US

(21) Appl. No.: 11/891,789

(22) Filed: Aug. 13, 2007

(51) Int. Cl. A01H 5/00

(52) U.S. Cl. PLT/255; 800/269

(57) ABSTRACT

A cultivar of Gardenia jasminoides, characterized by longevity and consistent production of seed, and G. thunbergia, recognized to have resistance to nematode damage, have been hybridized to combine the desirable characters of both species and facilitate production of improved cultivars.

### VARIETY DENOMINATION

The resulting Fl plant(s) are herewith identified as Gardenia Hybrid One.

### BACKGROUND AND SUMMARY OF THE INVENTION

Gardenia (Gardenia jasminoides) has been a favorite landscape plant in the warmer areas of the United States for more than a century. Popularity of the species has been encouraged, in large part, by the attractiveness of the plants and the allure of the pleasantly aromatic flowers. Additionally, the species is quite adaptable to various cultural methods and has modest to long-term life expectancy. There is a consistent lack of seed production in most cultivars despite frequent production of pollen and pistillate structures which leads to the presumption that most present-day cultivars have originated as asexual deviants. The species has few important cultural problems other than nematode infestation for which no cultivars of G. jasminoides are known to exhibit tolerance or resistance. Thus, a sizeable share of the plants produced for retail sale are now grafted onto stocks of G. thunbergia, the only recognized nematode-resistant species of the genus and the price to consumer must reflect this additional production cost. Unfortunately, this rootstock species is intolerant of freezing temperatures and, thereby, limits the range of successful, outdoor culture of grafted plants to zone 8 or warmer.

The discovery of a reliable seed-producing cultivar of G. jasminoides, i.e. “Star Bright” (U. S. Pat. No. 8,887) (SB), made possible horticultural improvement through controlled breeding. In 2004, cross pollinations on SB were made with pollen from G. thunbergia and, in due course, a population of four seedlings was obtained. These four plants have been reproduced repeatedly (asexually) by rooting cuttings and have now flowered and shown to have fertile pollen as well as being self-fertile. Flowers have the pleasing fragrance typical of the genus Gardenia.

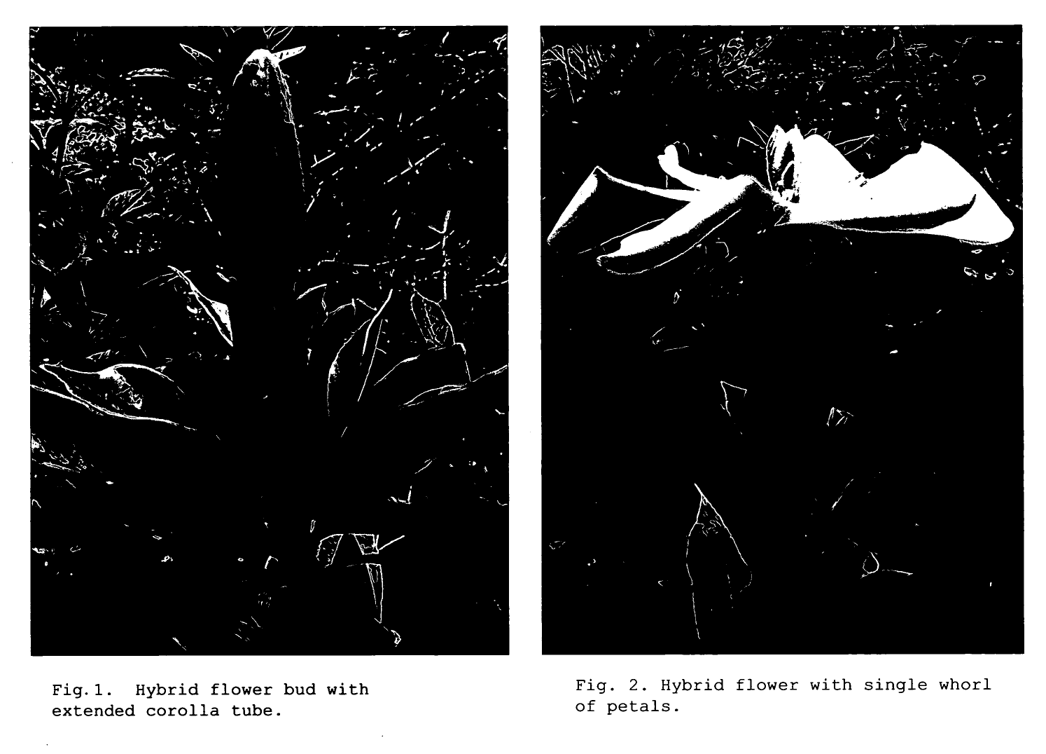

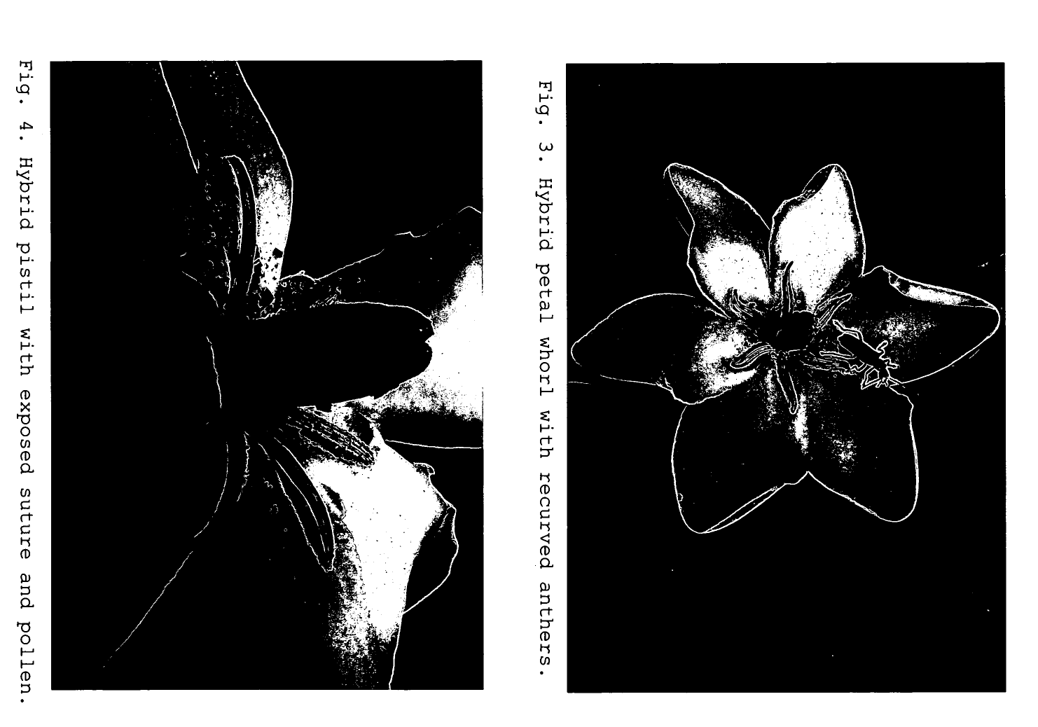

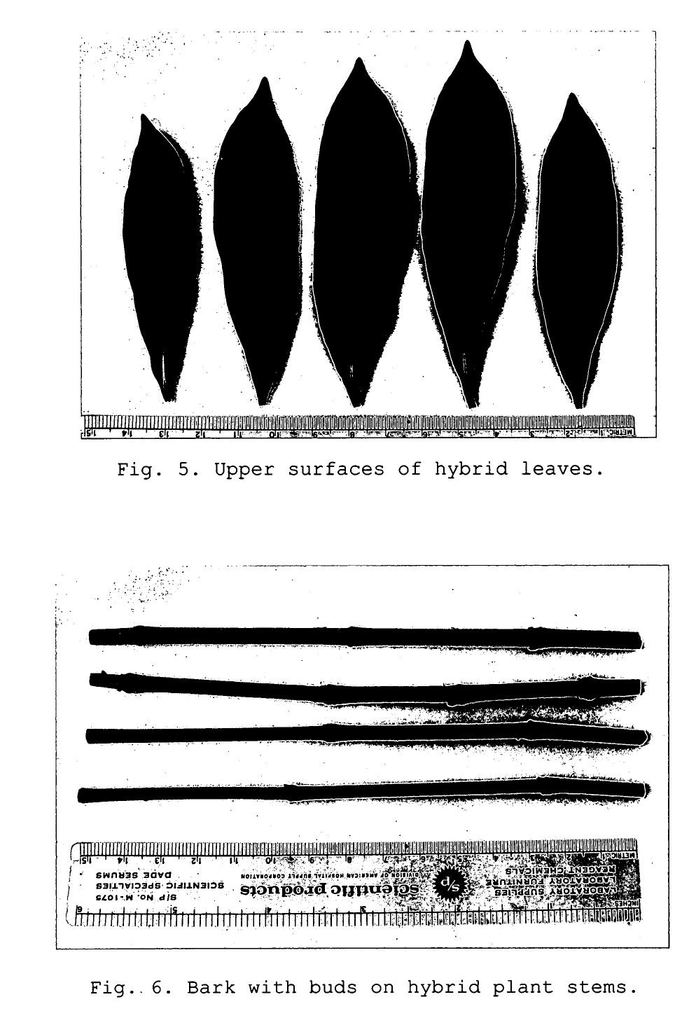

Flower formation in the pollen parent plant (G. thunbergia) is unique and typically begins with the appearance of a calyx tube nominally 12 mm in diameter and 10 cm in length with a ring of wings each about 3×10 mm at the top. Eventually, a split near the middle of this lengthy tube permits extrusion of the closed flower bud and subsequent extension of the corolla tube to approximately 5 cm with the bud at the apical end. Soon thereafter, a single whorl of (usually) eight white petals unfurls with the pistillate structure extending about 1 cm above the plane of the petals. The apical ends of the anthers, spaced alternately with the petals, partially separate from the petals at anthesis and remain upright thereafter attached in the upper throat of the corolla. As illustrated (see FIG. 1 and 2) flower development in the hybrid plant is quite different from that of the pollen parent and quite similar to that of the pistillate parent (SB). The calyx tube is absent with a much shorter (than G. thunbergia) corolla tube but still considerably longer (to 5 cm) than in the pistillate parent plant. Anthers are recurved outward after anthesis and remain attached in the throat of the corolla (FIG. 3). The pistillate structure protrudes a cm above the plane of the whorl of petals and appears to be divided in two parts with a strip of exposed tissue dividing the two halves as in S. B. There is abundant pollen produced by the hybrid (FIG. 4). The typical fragrance of the genus is retained in the hybrid and resembles that of the pistillate parent. Thus, the flower formation of the hybrid includes features from each of the two parent plants but not a predominance of characters from either. Leaves and stems of hybrid plants are indistinguishable from those of the pistillate parent (FIG. 5 and 6) while the pollen parent has leaves characteristically bellows-shaped. Mature plants may reach 1.5 m×1.5 m in size with moderately open branching as in the pistillate parent. Self-pollination in the hybrid plant has resulted in seed pod formation identical to that in SB and successful out-cross pollinations and seed production in several cultivars of G. jasminoides demonstrated.

The combined persistence of the original pistillate parent plant (SB), now more than thirty years old, and the acknowledged pest resistance of G. thunbergia, should contribute significantly to development of improved cultivars of gardenia.

FIG. 1. Side-view of hybrid bud and extended corolla tube

FIG. 2. Mature hybrid flower with a single whorl of petals

FIG. 3. Top view of hybrid petals after anthesis

FIG. 4. Side view of pistillate structure showing exposed suture

FIG. 5. Upper side view of hybrid plant leaves

FIG. 6. Bark/bud features of hybrid plant stems

### REFERENCES

Cook, A. A. 1994. Gardenia jasminoides var. Star Bright. U. S. Pat. No. 8,887. 6 p. Huxley, A., M. Griffiths and M. Levy. 1992. The New Royal Horticultural Society Dictionary of Gardening. The MacMillan Press Limited, London pp. 366-367.

I claim:

1. Hybridization of Gardenia jasminoides×G. thunbergia and the production of cultivars derived therefrom.
